(19) United States
(12) Patent Application Publication
(10) Pub. No.: US 2017/0325711 A1
(43) Pub. Date: Nov. 16, 2017

(54) METHOD AND APPARATUS FOR TESTING PLAUSIBILITY OF MEASUREMENT VALUES IN BODY COMPOSITION ANALYSIS

(71) Applicant: SECA AG, Reinach BL 1 (CH)

(72) Inventors: Björn JENSEN, Bergisch Gladbach (DE); Jan-Erik GROSSMANN, Hamburg (DE)

(21) Appl. No.: 15/667,903

(22) Filed: Aug. 3, 2017

Related U.S. Application Data
(63) Continuation-in-part of application No. 14/655,410, filed on Jun. 25, 2015.

(51) Int. Cl. A61B 5/053; A61B 5/00; G01G 19/50

(52) U.S. Cl. CPC A61B 5/0537; A61B 5/4875; A61B 5/4872; A61B 5/0535; G01G 19/50

(57) ABSTRACT

A method and apparatus for testing the plausibility of electric impedance measurement values. The measurement values are determined during the measurement of the bioimpedance of a person. Real parts and imaginary parts of the impedance measurement values are determined for a plurality of different frequencies and are localized in a complex representation plane. The representation plane is defined by a coordinate axis for the imaginary part and a coordinate axis for the real part. The localization of the measurement values in the complex representation plane is compared to a desired profile, and the measurement values are adjudged to be not plausible if a predefinable deviation from the desired profile is exceeded.

### CROSS-REFERENCE TO RELATED APPLICATIONS

The present application is a Continuation-In-Part Application of U.S. patent application Ser. No. 14/655,410, filed Jun. 25, 2015, which claims priority of a 371 of International application PCT/DE2013/000060, filed Jan. 29, 2013; the priority of these applications is hereby claimed and these applications are incorporated herein by reference.

### BACKGROUND OF THE INVENTION

The invention pertains to a method and an apparatus for testing the plausibility of electrical impedance measurements acquired during a measurement of the bioimpedance of a person.

The measurement of the electrical impedance of a person is typically done by the use of a so-called “body composition analyzer” (BCA) to obtain information on the composition of the body of the person in question. In the normal case, the percentages by weight of muscle mass, bone, fat, and water are determined. When these types of measurement methods are carried out, the person frequently is positioned on a measuring apparatus, standing with each foot on one of a pair of electrodes. Hand electrodes are also gripped or contacted manually.

The quality of the measurement values in question and thus also the quality of the information derived from them concerning the composition of the body depend on many different interference factors. These are, for example, body posture, the quality of the contact with the electrodes, and the positioning of the electrodes relative to the body.

In addition, there is often the problem that the person in question does not stand still during the performance of the measurement but rather moves around instead. This has the result that, when multiple measurement values are acquired in sequence, both accurate and inaccurate measurement values are obtained.

When measurements of the electrical impedance in question are to be carried out, the goal is to manage with the shortest possible measurement time and to take into account only qualitatively “good” measurement values. With the methods known so far, this cannot be done in a completely satisfactory manner.

One possible way of testing measurement values for their plausibility is to compare the recorded values with a reference. Such a comparison in the area of bioimpedance measurement has already been taught by Kun (U.S. Pat. No. 5,807,272) and Chetham (US 20100168530A1), who propose a Cole-Cole diagram as reference. In a Cole-Cole or Nyquist diagram, an impedance is graphed as a frequency-dependent locus in the complex plane. The goal of the comparison of the measurement value with the reference in Kun's teaching is to derive a diagnosis such as a circulatory disorder of body parts and/or transplanted tissue and, in Chetham's teaching, it is merely to obtain information for the operator of the apparatus concerning the possibility that the measurement does not conform to the Cole-Cole model, such conformity being necessary for a sufficiently accurate determination of the intersections with the real-part axis, i.e., the R₀ and R_{∞} values. Measurement values which deviate from the reference in Kun's and Chetham's teaching must be evaluated by a human being before a decision can be made concerning the action to carried out next. It is known that the effects of statistical error on a measurement can be taken into account by calibration.

### SUMMARY OF THE INVENTION

The goal of the present invention is to define a measurement method in such a way that, after an automatic plausibility test, incorrect individual measurement values caused by the above-described factors, especially the dynamic ones, are no longer taken into account in the following process; the measurement and the values derived from it thus become more accurate in their totality without the need to interrupt the measurement.

This goal is achieved according to the invented method in that the real parts and imaginary parts of the impedance measurement values are determined for a plurality of different frequencies and compared with respect to their localization in a complex representation plane defined by a coordinate axis for the imaginary part and a coordinate axis for the real part with a nominal curve, and in that the measurement value is considered implausible if it exceeds a predefinable deviation from the nominal curve.

Another goal of the invention is to implement an apparatus in such a way that, after a series of measurement values has been recorded, an automatic plausibility test of the measurement values can be conducted in order to correct the measurement result by eliminating errors occurring as a result of dynamic factors in particular. This goal is achieved according to the invention in that the apparatus according to the invention, which can be implemented as, for example, a Body Composition Analyzer (BCA), comprises a comparison unit, by means of which the recorded measurement values can be compared with a reference.

The method according to the invention can be visualized graphically by plotting a nominal curve, which shows a typical positioning of the real and imaginary parts for all frequencies between zero and infinity in the complex plane. If the distance between the real part and/or the imaginary part of a concrete measurement value and the point on the nominal curve belonging to its measurement frequency exceeds an allowable maximum with respect to absolute value and/or phase, the measurement value in question is not plausible.

Not only is the geometric course of the nominal curve known, but each point on this curve can also be uniquely assigned to a measurement frequency. As a result, it is possible to determine, with respect to absolute value and phase, the deviation of a measurement value which has been obtained at a concrete measurement frequency from the associated point on the nominal curve.

With respect to the performance of the method according to the invention, what is envisioned in particular is not to use a nominal curve unchangeably localized in the complex number plane but rather to check, on the basis of the concretely determined measurement values, whether these values lie on a curve which corresponds in shape to the nominal one. During the performance of the method, therefore, it is possible, for example, to interpolate between measurement values and to determine the curve which thus results. This curve is then compared with respect to shape and frequency-dependent positioning of the individual measurement values with the originally defined nominal curve.

It is irrelevant in particular whether or not the curve specified by the determined measurement values is shifted, for example, versus an expected curve shape with respect to the real parts and/or the imaginary parts or whether a different scaling occurs. The essential point is that there be agreement with the predetermined curve shape.

In particular, it is envisioned that, during the performance of the measurement, a frequency-dependent series of measurements is conducted, which determines individual measurement values at different measurement frequencies and stores them. These measurement frequencies extend from very small values to very large values, as large as can be technically realized. For the later evaluation, only the measurement values found to be plausible are used.

A typical performance of the method is defined in that a section of a circle in the complex plane is used as the nominal curve.

According to a typical measurement procedure, it is provided that the frequency is changed from a minimum value to a maximum value during the course of the measurement.

It is envisioned in particular that the minimum value is a frequency close to zero and the maximum frequency is as high as possible enabled by the hardware. An advantageous frequency range to conduct the bioimpedance measurements extends from 0.1 kHz to 10,000 kHz. It has been found especially advantageous to use frequencies in the range of 1 kHz to 1000 kHz.

To ensure the most accurate possible use of the nominal curve, it is proposed that at least three measurement values be determined.

It has been found especially advantageous to determine at least eight measurement values.

An apparatus according to the invention comprises the components necessary to implement the method according to the invention. In particular, an apparatus according to the invention has, in an advantageous embodiment, a function block by means of which a measurement signal at discrete measurement frequencies in the form of voltage and current values can be converted to a complex impedance. A comparison unit can compare this impedance with a reference. According to the invention, the comparison can be carried out completely in software, in hardware, or in a combination of hardware and software. The reference can be loaded from a memory or can be generated from a current measurement series. For the comparison with a reference generated from a measurement series, the comparison unit can be used to extract several impedance values from the measurement series, from which a reference curve can be produced by the use of a basic formula. For each individual measurement value, it is then possible, in the knowledge of the measurement frequency in question, to determine the deviation from the reference with respect to absolute value and phase.

On the basis of the defined tolerance band for absolute value and/or phase of the impedance values around the reference, measurement values which are outside the tolerance band can be adjudged implausible. These values can be discarded automatically, so that outliers in the measurement values can be excluded from a determination of the components of an equivalent circuit which can be implemented on a processor or controller, as a result of which the accuracy of the measurements is increased.

The various features of novelty which characterize the invention are pointed out with particularity in the claims annexed to and forming part of the disclosure. For a better understanding of the invention, its operating advantages, and specific objects attained by its use, reference should be had to the drawings and descriptive matter in which there are illustrated and described preferred embodiments of the invention.

### BRIEF DESCRIPTION OF THE DRAWING

In the drawing:

FIG. 1 shows a perspective view of an apparatus according to the invention for measuring a bioimpedance;

FIG. 2 shows a perspective view of hand electrodes of an apparatus according to the invention;

FIG. 3 shows a block circuit diagram of an apparatus according to the invention for measuring a bioimpedance;

FIG. 4 shows an equivalent electrical circuit diagram of the human body during the measurement of the bioimpedance;

FIG. 5 shows a formula for calculating the complex impedance of a “constant-phase element”;

FIG. 6 shows a diagram of bioimpedance measurement values and a reference curve in the complex number plane;

FIG. 7 shows a diagram illustrating the determination of a fitted curve by means of support points taken from a series of measurement values;

FIG. 8 shows a diagram illustrating a comparison of the existing measurement values with calculated values based on the fitted curve of FIG. 7;

FIG. 9 shows a graph of the deviations of the absolute values of measurements of a measurement series from associated points on the fitted curve and a check of whether or not the measurement values are within a predefined tolerance band;

FIG. 10 shows another graph illustrating phase deviations of measurements of a measurement series from the associated points on the fitted curve and the corresponding check of whether the measurement values are within a predefined tolerance band; and

FIG. 11 shows a flow chart of a method according to the invention for testing the plausibility of measurement values.

### DETAILED DESCRIPTION OF THE INVENTION

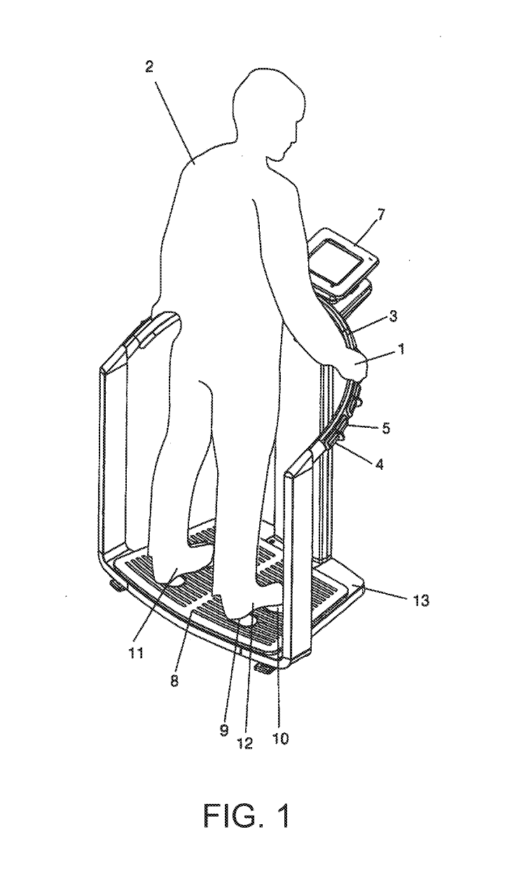

FIG. 1 shows an apparatus according to the invention for measuring a bioimpedance in an advantageous embodiment. A schematically illustrated person 2 has grasped the hand grip 3 in the area of the hand electrodes 4, 5 and is standing with his feet 11, 12 on the electrodes 9, 10 arranged in the area of a standing space 8. A scale 13 for measuring the weight of a person 2 can be integrated into the area of the standing space 8 of a body composition analyzer according to the invention.

A user interface 7, which can be configured as a view screen, for example, can be arranged in the viewing direction of the person 2.

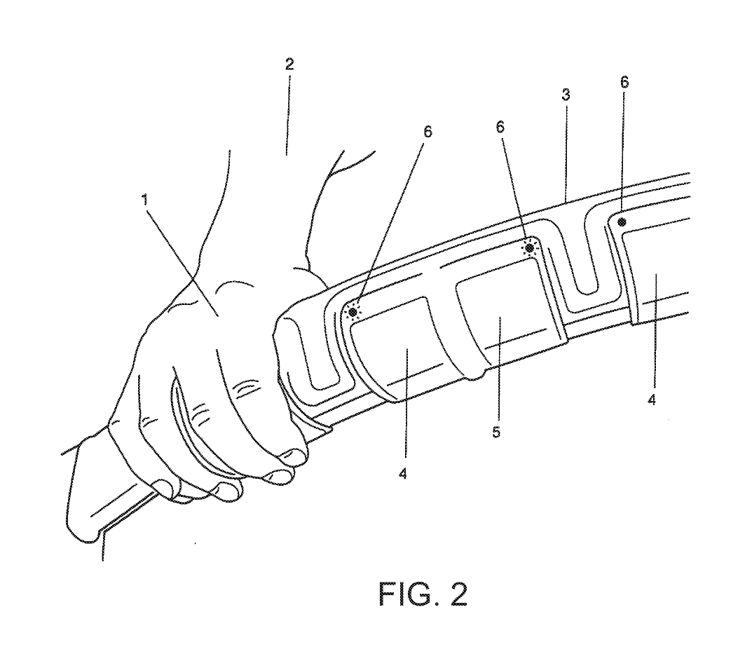

FIG. 2 shows a perspective diagram of an advantageous embodiment of a handgrip 3 of an apparatus according to the invention. The hand 1 of a person 2 to be measured grasps a pair of electrodes 4, 5. Several electrode pairs 4, 5, can be arranged in the area of the handgrip 3. In the area of the electrodes 4, 5, indicator lights 6 can be arranged, which can be configured as light-emitting diodes, for example, and which can indicate through a green or red color that, for example, the hand 1 has been correctly or incorrectly positioned.

Instead of a pair of electrodes, a single electrode per hand is also conceivable.

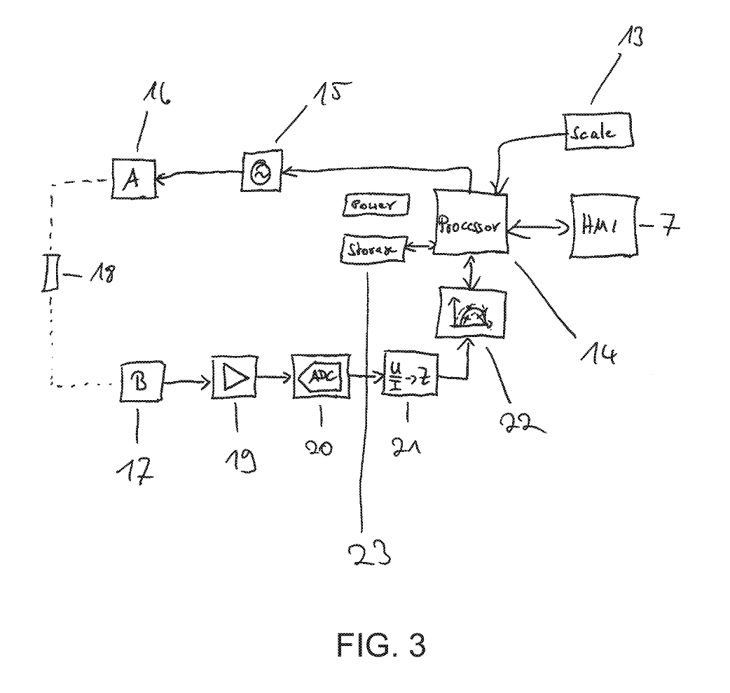

FIG. 3 shows a portion of the relevant blocks of a block circuit diagram of an advantageous embodiment of an apparatus according to the invention. A processor 14 can drive a signal generator 15, which can generate an electrical signal of a defined frequency ω. This signal can be applied to an electrode A 16 for the measurement of a bioimpedance. By means of appropriate wiring (not shown), several electrodes can be connected to one signal generator 15, as a result of which the signal can be conducted from various electrodes through the object to be measured. The signal processing of an apparatus according to the invention begins at a second electrode B 17, at least one of which is present. The goal of a measurement is to determine the complex bioimpedance 18 between the selected electrodes 16, 17. The current and voltage signals which can be measured at the electrode B 17 can be varied by an analog circuit 19 in such a way that the metrological requirements in question can be fulfilled to a sufficient degree. For example, in an advantageous embodiment, a combined filter and amplifier circuit is envisioned. By means of an analog-digital converter 20, the analog signals can be converted digital values. A function block 21 can determine the complex impedances Z at the discrete measurement frequencies ω. In a comparison block 22, the impedance values recorded and calculated in one measurement can be compared with a reference curve. The reference curve can be loaded individually from a memory 23 for various parameters and measurement situations, or it can be generated from the current measurement data. In addition, the comparison block 22 can be used to determine the distance of an impedance value acquired from a measurement from the corresponding point on the reference curve with respect to absolute value and phase and to compare it with defined tolerance values. Measurement values which lie outside a defined tolerance can be adjudged implausible and can be discarded by the comparison block 22. The remaining set of impedance values adjudged plausible can be sent for further processing to the processor 14, where additional steps of the body composition analysis can be carried out.

In the advantageous embodiment shown here, it is also possible to connect to the processor 14 an integrated scale 13 and a user interface 7, which can be configured as, for example, a view screen and an input device.

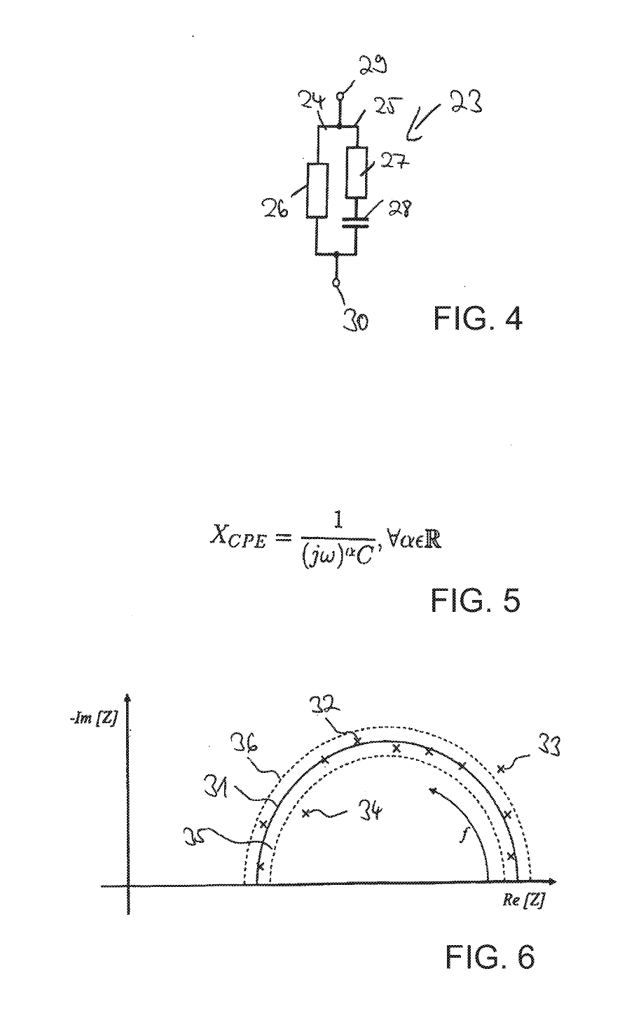

FIG. 4 shows an equivalent electrical circuit diagram 23 for the human body during the measurement of the bioimpedance. The equivalent circuit diagram comprises two parallel branches 24, 25, wherein a resistor 26 is present in the branch 24, and a resistor 27 and a capacitor 28 are connected in series in the branch 25. The branches 25, 26 are brought together at the terminals 29, 30.

The capacitor 28 in the equivalent circuit diagram 23 is shown only as an example. In particular, it is also envisioned that, instead of the capacitor 28, a so-called “constant-phase element” could be used.

When DC voltage is applied to the terminals 29, 30, only the resistor 26 is electrically active because of the constant-phase element 28.

When AC voltage is applied to the terminals 29, 30, the resistors 26, 27 become connected progressively more in parallel with increasing frequency. Because of the complex impedance of the “constant-phase element” 28, however, a phase shift is observed.

The constant-phase element behaves largely like a non-ideal capacitor.

The complex impedance of the constant-phase element is obtained from the formula of FIG. 5.

FIG. 6 shows the impedance of the equivalent circuit diagram 23 within the complex number plane in the form of a locus 31. It can be seen that, when the DC current falls and thus for a frequency of zero, the impedance has only a real part, wherein this is determined by the resistor 26. As the frequency increases, the real part decreases, and the absolute value of the imaginary part increases at first. At an infinite frequency, the absolute value of the imaginary part of the impedance returns to zero again, and the impedance has only a real part, which results from the parallel connection of the resistors 26, 27.

The exact semi-circular locus 31 shown is obtained under consideration of the constant-phase element 28 in the equivalent circuit diagram 23. If, instead of the constant-phase element 28, the previously mentioned capacitor is used, then the locus 31 has the form of a shorter section of a circle.

The crosses 32, 33, 34 represent the impedance values, based on a measurement, at the discrete measurement frequencies. The broken lines 35, 36 define a tolerance band around the reference locus 31. The measurement values 32 which lie within the tolerance area defined by the lines 35, 36 are considered plausible. The measurement values 33, 34 which lie outside the tolerance band are discarded.

The nominal curve can be determined geometrically, for example. The section of the circle in question is determined on the basis of at least two, preferably three, support points from the measurement series, and then the values of the components of the equivalent circuit diagram are determined. The intersection between the locus and the real axis at the greatest distance from the imaginary axis corresponds to the value of the resistor 26. The intersection between the locus and the real axis at the shortest distance to the imaginary axis corresponds to the value of the parallel connection of the two resistors 26, 27. The values of these two resistors are defined by this means.

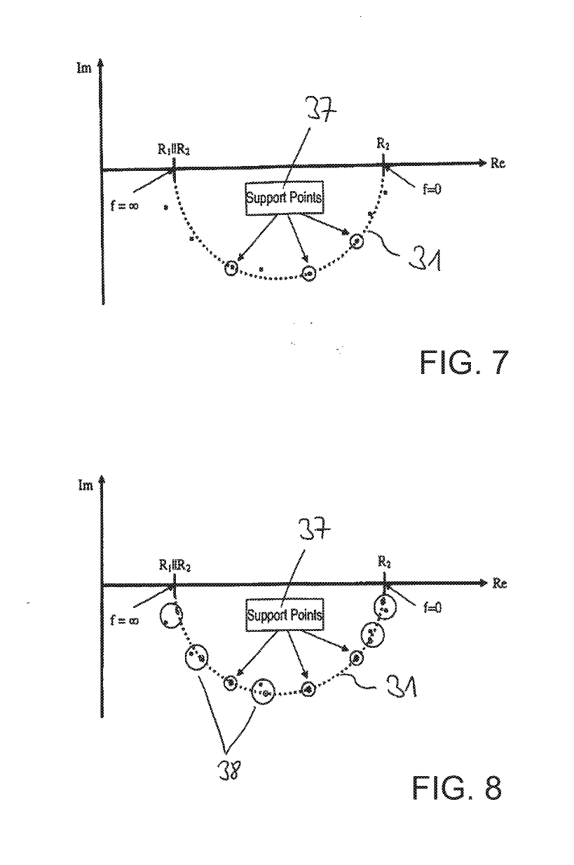

FIG. 7 shows the generation of a reference curve 31 from a measurement series. Three measurement values, which are used as support points 37 for the calculation or interpolation of an exact semi-circle 31, are selected from the essentially semicircular distribution of the measurement values 32, 33, 34. The generation of the interpolated reference semicircle 31 can also be carried out from a larger number of measurement values selected as support points 37. There are various methods which can be used to determine the measurement values of a measurement series which are to be used as support points 37. Envisioned are a random selection; a previously defined selection based, for example, on the frequency or on the position within the measurement series; or a selection based on a more extensive optimization process, which determines the support points of the curve from which, on average, the shortest distance to all measurement values of a measurement series is present.

FIG. 8 shows for comparison, in the large circles 38, the measured impedances and the corresponding impedances based on the reference curve at the discrete measurement frequencies.

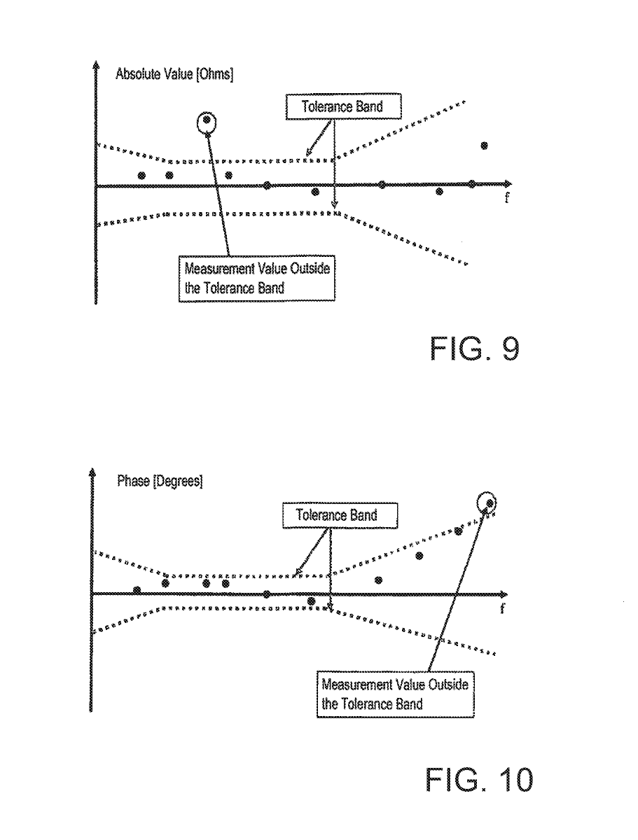

FIGS. 9 and 10 show separate considerations of the absolute values and phases of the measured impedances and the tolerances defined in each case.

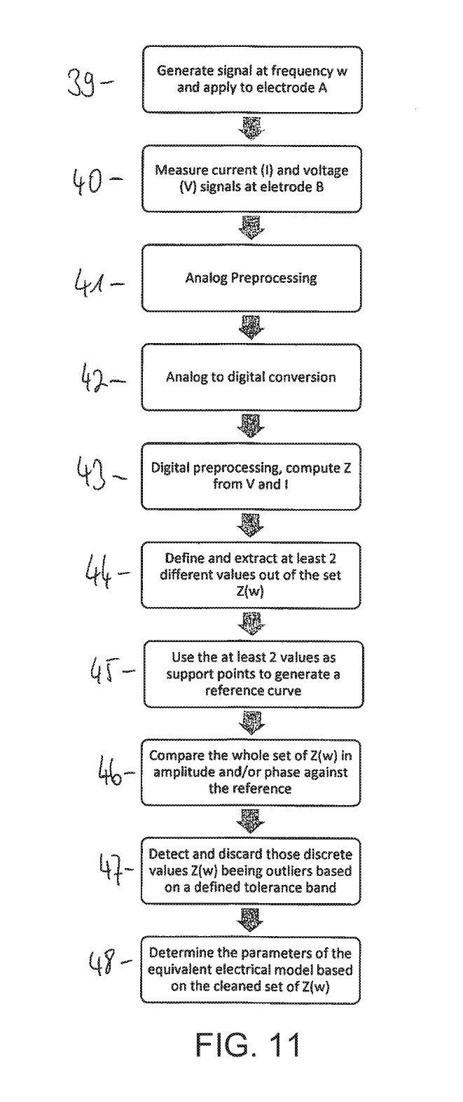

FIG. 11 shows a flow chart of a method according to the invention. The starting point is the performance of a measurement at various discrete frequencies, advantageously distributed over a wide frequency range, represented by steps 1/39 and 2/40. The measurement advantageously consists of at least three, especially advantageously of eight, measurement values at various frequencies.

In the next step 41, the individual measurement signals are subjected to analog signal processing before they are digitized 42.

The digital measurement data are possibly subjected to further digital processing and the impedances at the discrete measurement frequencies determined from the measured values of current and voltage in step 43.

In the next step 44, the method according to the invention consists of selecting, from the totality of the impedances based on the current measurement, at least two values, which are used as support points 37 for generating a reference curve 31. The selection of the support points 37 can be implemented, for example, by a random selection; by a previously defined selection based, for example, on the frequency or the position within the measurement series; or by a selection based on a more extensive optimization process, which determines the support points of the curve 31 from which, on average, the shortest distance to all measurement values of a measurement series is present. In an advantageous embodiment of the method according to the invention, the form of the reference curve 31 is defined by a mathematical formula corresponding to the overall impedance of the electrical equivalent circuit diagram of a human body or body part 23, which is especially advantageous for a body composition analysis.

After the selection of the support points 37, the next step 45 is to generate the reference curve 31 as described above. Alternatively, it is also conceivable that a predefined reference curve 31 could be loaded from a memory 23, this curve possibly being modifiable on the basis of the current measurement series or other input parameters such as, for example, the age, height, or weight of the person 2 to be measured.

In the next step 46, the distance of all the complex impedances from the reference curve 31 is determined. The can be done with respect to absolute value and/or phase on the basis of the locus 31 shown in FIG. 6 or separately, as shown in FIGS. 7 and 8. The distances are compared with the defined tolerances.

In the next step 47, the measurement values which do not lie within the tolerance band are detected and discarded.

In the next step 48, the ‘cleaned’ measurement data are sent to the additional processing phase. In an advantageous embodiment of the implementation of the method according to the invention, this additional processing consists of the interpolation of a locus 31 with the smallest deviation from the distribution of the measurements values corrected by the above-described method. Based on the locus 31, the parameters 26, 27, 28 of the equivalent circuit diagram 23 are then determined, which, for example, can now be used to determine the composition of the body of the person 2.

The apparatus according to the invention and the method according to the invention thus make it possible to arrive at a more accurate determination of the components 26, 27, 28 of the equivalent circuit diagram 23 and thus an increased accuracy of the analysis of the body composition when used for the purpose of BCA in comparison to an apparatus or a method without plausibility testing of the measurement values.

While specific embodiments of the invention have been shown and described in detail to illustrate the inventive principles, it will be understood that the invention may be embodied otherwise without departing from such principles.

We claim:

1. A method for automated plausibility testing of measurement values of a bioimpedance measurement, comprising the steps of:
recording a frequency-dependent measurement series to determine individual impedances at discrete frequencies of the measurement;
selecting support points based on the acquired impedances;
using the selected support points to generate a reference curve;
determining the deviations of the individual impedances of the measurement series from the reference curve with respect to absolute value and/or phase;
comparing the deviations with a defined tolerance; and
detecting and discarding measurement values that do not lie within the defined tolerance band.

2. The method according to claim 1, wherein at least two impedances are selected as support points.

3. The method according to claim 1, wherein the selection of the support points is made on a random basis.

4. The method according to claim 1, wherein the selection of the support points is made as a function of an associated frequency or a position of a value in the measurement series.

5. The method according to claim 1, wherein the selection of the support points is based on an optimization process, which selects the support points that make possible the reference curve with a smallest average deviation from all measurement values.

6. The method according to claim 1, wherein a procedure for generating the reference curve is based on a frequency-dependent impedance of a parallel circuit of a resistor with a series circuit of a resistor and a constant-phase element.

7. The method according to claim 1, wherein at least three measurement values are acquired.

8. The method according to claim 7, wherein at least eight measurement values are acquired.

9. An apparatus for automated plausibility testing of measurement values of a bioimpedance measurement, comprising: a comparison block, that compares acquired measurement values with a reference, and discards the values that exceed a defined deviation with respect to absolute value and/or phase.

10. The apparatus according to claim 9, wherein a measurement series for impedance determination is carried out over a wide frequency range, which extends from very small frequencies close to 0 Hz to frequencies which are as high as technically realizable.

11. The apparatus according to claim 10, wherein the measurement range extends from 100 Hz to 10 MHz.

12. The apparatus according to claim 9, wherein the reference is selected from the recorded measurement values according to a technical procedure.

13. The apparatus according to claim 12, wherein the reference is generated based on a frequency-dependent impedance of an electrical equivalent circuit diagram of biological tissue, which consists of a parallel circuit of a resistor with a series circuit of a resistor and a constant-phase element.
